SEQUENCE LISTING (2 sequences)

SEQ ID NO: 1 - protein, 143 aa, Artificial sequence
  DeltaNS1
MGSSHHHHHHSSGLVPRGSHMASMTGGQQMGRGSSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTALEHHHHHH

SEQ ID NO: 2 - DNA, 432 bp, Artificial sequence
  Encodes the protein of SEQ ID NO 1
atgggcagcagccatcatcmtywtcatcacagcagcggcctggtgccgcgcggcagccatatggctagcatgactggtggacagcaaatgggtcgcggatccagccatcataatacccgtgaaggttaccgtacccaaatgaaaggtccgtggcatagcgaagaactggagattcgttttgaagagtgcccgggtaccaaggtgcacgttgaggagacctgcggtacccgtggcccgagcctgcgtagcaccaccgcgagcggtcgtgtgattgaggaatggtgctgccgtgagtgcaccatgccgccgctgagcttccgtgcgaaagacggttgctggtacggcatggaaatccgcccgcgcaaggagccggaaagcaatctggttcgtagcatggttaccgcgctcgagcaccaccaccaccaccactga

(19) United States
(12) Patent Application Publication
(10) Pub. No.: US 2021/0388409 A1
(43) Pub. Date: Dec. 16, 2021

(54) PPROCESS FOR PRODUCING A RECOMBINANT FRAGMENT OF THE C-TERMINAL REGION OF THE FLAVIVIRUS NONSTRUCTURAL SOLUBLE PROTEIN NS1, PURIFICATION PROCESS, PRODUCT, USE OF THE PRODUCT, METHOD OF DETECTION AND METHOD OF DIAGNOSIS

(71) Applicant: INSTITUTO BUTANTAN, São Paulo (BR)

(72) Inventors: Luís Carlos De Souza FERREIRA, São Paulo (BR); Lennon Ramos PEREIRA, São Paulo (BR); Mônica Josiane Rodrigues De JESUS, Diadema (BR); Luciana Cezar De Cerqueira LEITE, São Paulo (BR); Alex Issanu KANNO, São Paulo (BR); Viviane Maimoni GONÇALVES, São Paulo (BR)

(21) Appl. No.: 17/272,618

(22) PCT Filed: Sep. 2, 2019
(86) PCT No.: PCT/BR2019/050362

§ 371 Date: Mar. 1, 2021

(30) Foreign Application Priority Data
Aug. 31, 2018 (BR) 102018067345-9

(51) Int. Cl. C12P 21/02; C12N 15/70; G01N 33/569; C07K 14/18

(52) U.S. Cl. CPC C12P 21/02; C07K 14/18; G01N 33/569; C12N 15/70

(57) ABSTRACT

The present invention is within the Molecular Biology and Biochemistry and Biotechnology fields. More specifically, the present invention describes a process for producing the recombinant fragment of the c-terminal region of the flavivirus NS1 non-structural soluble protein and the recombinant protein (Zv-ΔNS1) in large scale. The product of the invention has advantageous characteristics as a result of the process for obtaining the same, notably regarding folding and the immunological characteristics suitable for the development of serological tests to detect Zika virus. There are also described a purification process, its use and a method of detecting interaction, and a method of diagnosing diseases caused by a flavivirus.

### FIELD OF INVENTION

The present invention resides in the fields of Molecular Biology and Biochemistry and Biotechnology. More specifically, the present invention describes a large-scale production process for a recombinant fragment of the c-terminal region of the flavivirus NS1 non-structural soluble protein, including the Zika virus (Zv-ΔNS1) (e.g., SEQ ID No 1), the product obtained by the process, the use thereof and a method of detecting interaction and a method of diagnosing diseases caused by flavivirus. The product of the invention has advantageous characteristics as a result of the process for obtaining the same, notably with regard to folding and the appropriate immunological characteristics for the development of serological tests for the detection of the Zika virus.

### BACKGROUND OF THE INVENTION

The outbreak of the Zika virus (ZIKV) represents a serious problem for populations living in or visiting endemic areas and a major challenge to the Brazilian health system. Among the main difficulties encountered in dealing with the ZIKV outbreak is the complexity of designing a specific serological diagnosis due to the extensive cross-reactivity of antibodies produced in people infected with different flaviviruses, especially the dengue virus (DENV) and ZIKV. One approach to this limitation is the development of more specific methods of diagnosis, such as the use of recombinant proteins or fragments derived therefrom, which provide the distinction between the antibodies produced after infection by ZIKV and DENV.

The production of recombinant proteins in Escherichia coli adapted in the laboratory is one of the most common approaches used to investigate the use of an antigen for a vaccine, a therapeutic target, or in functional studies. The exploitation of these organisms for exhaustive production of an abundant amount of a recombinant protein of interest often leads to the formation of inclusion bodies, that is, cytoplasmic aggregates of insoluble proteins. These inclusion bodies are difficult to work with and are generally avoided, as they require denaturation, purification, and renaturation processes, which usually lead to low recovery of the protein of interest. Thus, it is desirable to carry out the production of soluble recombinant proteins, however, this is one of the biggest obstacles in the academic and industrial environment.

Several factors can influence the production of recombinant proteins in their soluble form, which involve the host strain, characteristics related to the plasmid, and characteristics related to the cultivation process per se, such as the culture conditions and compositions of the culture media. In bioprocesses, the best conditions are determined based on the final yield considering the biomass, the product and the time spent that may not be the same for each prioritized parameter and, therefore, require practical determination.

The outbreak of the Zika virus (ZIKV) caused great apprehension in the populations of several countries and much effort was concentrated on the development of a differential diagnosis for ZIKV infection, particularly in regions endemic for the dengue virus (DENV).

The recent mandatory testing coverage for ZIKV provided by health insurance companies (medical insurance), in vitro fertilization operations, and blood transfusions require the development of more specific serological diagnostic tests. Although there are some options available, the development of more reliable and more specific serological tests is still desired. NS1-based serological tests have been particularly successful in differentiating between ZIKV and DENV infections. Nevertheless, the observations showed that a recombinant fragment from the C-terminal portion of the NS1 protein (Zv-ΔNS1) can improve the specificity of ZIKV serological tests. While the production on a laboratory scale of Zv-ΔNS1 will lead to product validation per se, the challenge is to increase the production scale to sustain a continuous demand from the health system.

Patent application BR 10 2016 011318 filed on May 18, 2016 on behalf of the University of Sao Paulo (USP) describes a recombinant antigen derived from the ZIKV non-structural protein 1 (deltaN-NS1) capable of differentiating, from a specific form, antibodies generated after ZIKV infection and differentiate them from antibodies generated after infection with the dengue virus (DENV) or other flaviviruses. Additionally, the patent application BR 10 2016 011318 describes the assembly of serological diagnostic kits for the detection of ZIKV in different technological application platforms, as well as its use in the differential diagnosis of individuals infected by ZIKV.

On the other hand, the present invention provides a solution for high yield production of the Zv-ΔNS1 soluble recombinant protein, in a single batch, sufficient for the production of three hundred thousand ELISA tests.

From what can be inferred from the researched literature, no documents were found anticipating or suggesting the teachings of the present invention, so that the solution proposed here, in the eyes of the inventors, has novelty and inventive activity in view of the state of the art.

### SUMMARY OF THE INVENTION

The present invention aims to solve the constant problems in the state of the art, by means of a process for obtaining the recombinant fragment of the c-terminal region of the flavivirus NS1 non-structural soluble protein. In one embodiment, it may be of the soluble recombinant protein comprising the amino acid sequence of SEQ ID No 1.

In a first object, the present invention discloses a process for the production of a recombinant fragment of the c-terminal region of the flavivirus NS1 non-structural protein, comprising the following steps:

(i) obtaining a host cell comprising an expression system of the flavivirus NS1 non-structural soluble protein;

(ii) cultivating the host cells at a temperature of 16 to 30° C. for 8 to 24 hours.

In a second object, the invention presents a process of purification of the recombinant fragment of the c-terminal region of the flavivirus NS1 non-structural soluble protein, produced by the process described herein comprising the steps of:

(a) Cell lysis;

(b) Centrifugation;

(c) Metal affinity chromatography; and

(d) Molecular exclusion chromatography for the separation of the recombinant protein from the c-terminal region of the Flavivirus NS1 non-structural protein.

In a third object, the present invention presents the product obtained by the process.

In a fourth object, the present invention presents the use of the product for the preparation of a kit to identify the interaction of the recombinant fragment of the c-terminal region of the NS1 non-structural protein with antibodies against flavivirus.

In a fifth object, the present invention presents a method of detecting the interaction between antibodies generated after infection by ZIKV, DENV, or other flaviviruses comprising the steps of:

(i) contacting a recombinant fragment of the c-terminal region of the flavivirus NS1 non-structural soluble protein, obtained by the process with an ex vivo biological fluid sample;

(ii) providing conditions for said recombinant protein to bind to a specific antibody against the same, present in said sample;

(iii) visualizing or detecting the interaction of said recombinant protein with said antibody;

(iv) comparing the results of said visualization and/or detection with a standard.

In a sixth object, the present invention presents a method of diagnosing diseases caused by flavivirus comprising the steps of:

(i) contacting a recombinant fragment of the c-terminal region of the Flavivirus NS1 non-structural protein, with a biological fluid sample;

(ii) providing conditions for said recombinant protein to bind to a specific antibody against the same, present in said sample;

(iii) visualizing the interaction of said recombinant protein with said antibody; and

(iv) diagnosing whether the biological fluid sample is infected or has already been infected with a flavivirus or not.

These and other objects of the invention will be immediately valued by those skilled in the art and by companies with interests in the field and will be described in sufficient detail for their reproduction in the description below.

### BRIEF DESCRIPTION OF THE DRAWINGS

In order to better define and clarify the content of this patent application, the following figures are presented:

FIG. 1 shows the evaluation of culture media for E. coli Arctic Express (DE3) and BL21 (DE3) strains producing Zv-ΔNS1 and comparisons between the strains. After transformation, three clones were cultured, induced with IPTG, overnight, and analyzed for SDS-PAGE and Western blot. In this first approach, the LB and TB culture media were tested (FIG. 1 A-B), after the induction and separation of the soluble and insoluble protein fractions, the Zv-ΔNS1 production was analyzed by SDS-PAGE (FIG. 10). A (Figure D1) compares the Zv-ΔNS1 production between strains, 5 μg of extracts of soluble proteins from six E. coli Arctic clones and three BL21 (DE3) clones were separated by SDS-PAGE and quantified.

FIG. 2 shows, by means of the SDS-PAGE result, the Zv-ΔNS1 protein present in whole E. coli cell extracts from the culture in a batch bioreactor with 6-L of culture medium.

FIG. 3 shows the experimental tests (open circles) and the three experiments for the optimized conditions (closed circles). The triangle refers to the optimized condition.

FIG. 4 shows the elution that was carried out with an imidazole increasing gradient. The arrow marks the elution peak of ΔNs1.

FIG. 5 shows the molecular exclusion chromatography of the fraction containing ΔNS1. The arrow marks the elution peak of ΔNs1.

FIG. 6 shows the purified protein obtained from the molecular exclusion chromatography, in an amount of 70 mg and with purity >95%.

FIG. 7 shows the antigenicity and specificity of the recombinant protein ΔNS1. Coomassie blue staining (left panels) and Western blots (right panels), obtained with 1 μg of purified ΔNS1 or complete Brazilian ZIKV NS1, and DENV2 NS1 (NGC lineage). Western blots were probed with an anti-His-Tag mAb (ZIKV proteins) or anti-DENV2-NS1. b ELISA reaction of human immune serum from individuals infected with DENV with the proteins DENV NS1, ZIKV NS1 and ΔNS1. c Reaction of the human serum sample ZIKV+DENV+with the proteins DENV NS1, ZIKV NS1, and ΔNS1 intact and previously denatured by heat.

### DETAILED DESCRIPTION OF THE INVENTION

The present invention discloses a large-scale process of production of the recombinant fragment of the c-terminal region of the flavivirus NS1 non-structural protein, a purification process, a large-scale process of production of the soluble recombinant protein and which can comprise the amino acid sequence of SEQ ID No 1, the product obtained by such processes, the use thereof and a method of diagnosing diseases caused by flavivirus. The process of the invention provides conditions to increase the production of the soluble protein in E. coli, preserving the immunological characteristics suitable for the development of serological tests.

In a first object, the present invention discloses a process for the production of a recombinant fragment of the c-terminal region of the flavivirus NS1 non-structural soluble protein, comprising the following steps:

(i) obtaining a host cell comprising a flavivirus NS1 non-structural protein expression system;

(ii) cultivating the host cells at a temperature of 16 to 30° C. for 8 to 24 hours.

In one embodiment of the process, the induction of NS1 non-structural protein expression is performed with an inducer.

In one embodiment of the process, the induction is performed with an inductor selected from IPTG and lactose.

In one embodiment of the process, the induction is performed with IPTG.

In one embodiment of the process, the induction is performed with 0.4 to 1 mM of the inductor.

In one embodiment of the process, the cultivation time is 10 to 12 hours for cultivation in a bioreactor.

In one embodiment of the process, the cultivation time is 18 to 24 hours for cultivation in a bottle.

In one embodiment of the process, the cultivation takes place in a medium that supports the growth of the host cell.

In one embodiment of the process, the cultivation occurs in LB, TB, and 2×HKSII medium.

In one embodiment of the process, the host cell is Escherichia coli.

In one embodiment of the process, the same is conducted under one or more of the following conditions:
temperature of 21° C.;
induction of the expression of the gene of interest made with 0.7 mM of IPTG;
host microorganism selected from Escherichia coli Arctic Express (DE3) and BL21 (DE3).

In one embodiment of the process, the host cell is BL21 (DE3).

In one embodiment of the process, the obtained product shows water solubility above 60% in relation to the total amount of soluble and insoluble protein.

In one embodiment of the process, the obtained product shows water solubility above 70% in relation to the total amount of soluble and insoluble protein.

In one embodiment of the process, the obtained product shows water solubility above 80% in relation to the total amount of soluble and insoluble protein.

In one embodiment of the process, the obtained product shows water solubility above 90% in relation to the total amount of soluble and insoluble protein.

In one embodiment of the process, the concentration of the obtained final product is greater than 0.5 g/L.

In one embodiment of the process of producing the recombinant fragment of the c-terminal region of the flavivirus NS1 non-structural soluble protein, the fragment has 70% identity to SEQ ID No 1.

In one embodiment of the process, the recombinant fragment of the c-terminal region of the flavivirus NS1 non-structural soluble protein has 80% identity to SEQ ID No 1.

In one embodiment of the process, the recombinant fragment of the c-terminal region of the flavivirus NS1 non-structural soluble protein has 90% identity to SEQ ID No 1.

In one embodiment of the process, the recombinant fragment of the c-terminal region of the flavivirus NS1 non-structural soluble protein has 95% identity to SEQ ID No 1.

In one embodiment of the process, the recombinant fragment of the c-terminal region of the flavivirus NS1 non-structural soluble protein has 99% identity to SEQ ID No 1.

In one embodiment of the process, the recombinant fragment of the c-terminal region of the flavivirus NS1 non-structural soluble protein is SEQ ID No 1.

In one embodiment of the process, the nucleic acid comprised in the host cell is SEQ ID No 2.

In a second object, the invention presents a process of purification of the recombinant fragment of the c-terminal region of the flavivirus NS1 non-structural protein, produced according to the process, comprising the steps of:

(a) cell lysis;

(b) centrifugation;

(c) metal affinity chromatography; and

(d) molecular exclusion chromatography for the separation of the recombinant protein from the c-terminal region of the flavivirus NS1 non-structural protein.

In one embodiment of the purification process, it is carried out under one or more of the following conditions:

(a) cell disruption was carried out in a continuous high-pressure homogenizer, at 600 bar and 1 L/min for 8 min;

(b) centrifugation at 17,000 g for 2 h;

(c) Ni⁺² charged metal affinity chromatography previously equilibrated with bis-tris propane buffer 10 mM pH 8.5, 0.5 M NaCl; and/or

(d) elution carried out with imidazole increasing gradient, followed by molecular exclusion chromatography.

In a third object, the present invention presents a product obtained by the process as described herein.

In one embodiment, the product obtained by the process, that is, the recombinant protein produced by the process of the invention has advantageous characteristics for use as a result of the process of obtaining the same, notably concerning to the folding of the protein that provided immunological characteristics suitable for the development of serological tests for the detection of flavivirus, e.g., Zika virus.

In a fourth object, the present invention presents the use of the product for the preparation of a kit to identify the interaction of the recombinant fragment of the c-terminal region of the NS1 non-structural protein with antibodies against flavivirus.

In a fifth object, the present invention presents a method of detecting the interaction between antibodies generated after infection by ZIKV, DENV, or other flaviviruses comprising the steps of:

(i) contacting a recombinant fragment of the c-terminal region of the flavivirus NS1 non-structural soluble protein, obtained by the process with an ex vivo biological fluid sample;

(ii) providing conditions for said recombinant protein to bind to a specific antibody against the same, present in said sample;

(iii) visualizing or detecting the interaction of said recombinant protein with said antibody;

(iv) comparing the results of said visualization and/or detection with a standard.

In a sixth object, the present invention presents a method of diagnosing diseases caused by flavivirus comprising the steps of:

(i) contacting a recombinant fragment of the c-terminal region of the flavivirus NS1 non-structural protein, with a biological fluid sample;

(ii) providing conditions for said recombinant protein to bind to a specific antibody against the same, present in said sample;

(iii) visualizing the interaction of said recombinant protein with said antibody; and

(iv) diagnosing whether the biological fluid sample is infected with a flavivirus or not.

In an embodiment of the diagnostic method, the diseases to be diagnosed are selected from: yellow fever; dengue 1, 2, 3, and 4, Zika virus, West Niles virus, St. Louis encephalitis virus, Murray Valley encephalitis virus, tick encephalitis, and others.

In one embodiment of the diagnostic method, the disease is the Zika virus.

### EXAMPLES

The examples shown herein are intended only to exemplify one of the countless ways of carrying out the invention, however without limiting the scope of the same.

### Example I—Evaluation of Zv-ΔNS1 Expression in E. coli Arctic and BI21 (DE3) Strains

Several parameters were tested for psychrophilic strains of E. coli Arctic grown at a very low temperature (11° C.), to favor the adequate folding of the recombinant protein.

The pET28a vector was used, which is a bacterial plasmid vector with the T7lac promoter that adds a hexahistidine tail in the N and C-terminal portion. It has a multi cloning site where the fragment of interest was cloned under the regulation of the T7 promoter and the lac operon operator. There is an E. coli origin of replication (f1) and a resistance marker to kanamycin antibiotic.

Originally the vector is about 5.4 kb, which with the addition of the sequence of interest under the XhoI and BamHI sites removes 34 base pairs and adds 304 bp of the specific sequence instead, resulting in a vector of ˜5.7 kb.

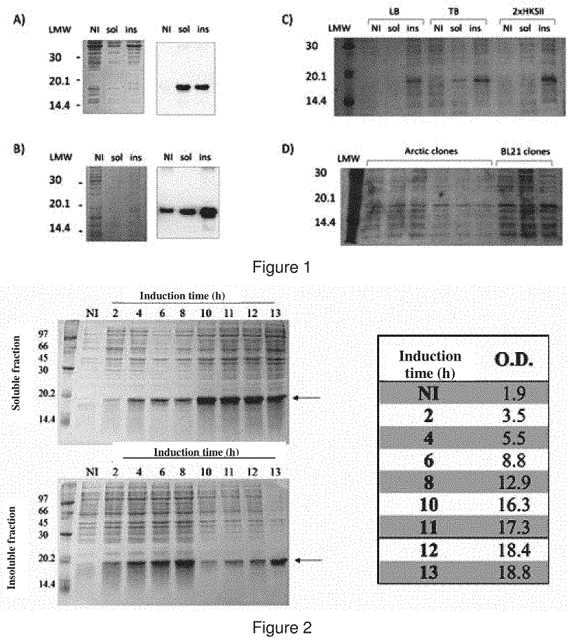

After transformation, three clones were cultured, induced with IPTG, overnight, and analyzed for SDS-PAGE and Western blot. In this first approach, the LB and TB culture media were tested (FIG. 1 A-B). No prominent production of Zv-ΔNS1 was observed in either culture medium in SDS-PAGE, however, a high production of Zv-ΔNS1 was present in Western blot when the TB culture medium was used, despite the majority of Zv-ΔNS1 be concentrated in the insoluble fraction (FIG. 1b). In addition, the strains grown in a low-temperature environment showed a low biomass yield (OD˜5 maximum).

BL21 (DE3) strain was also evaluated using three different culture media: LB, TB, and 2×HKSII. After induction and separation of the soluble and insoluble protein fractions, the production of Zv-ΔNS1 was analyzed by SDS-PAGE (FIG. 10). Both LB and 2×HKSII media showed Zv-ΔNS1 concentrated in the insoluble fraction but in different amounts. Despite this, TB culture medium provided the production of soluble Zv-ΔNS1 in sufficient quantity for the observation of a visible band on SDS-PAGE. In order to compare Zv-ΔNS1 production between strains, 5 μg of soluble protein extracts from six E. coli Arctic clones and three BL21 (DE3) clones were separated by SDS-PAGE and quantified (Figure D1). Despite some variations, BL21 (DE3) was clearly able to produce more protein than the Arctic strain, allowing Zv-ΔNS1 to be observed in SDS-PAGE, in addition to achieving greater biomass (OD˜7).

In order to ensure a greater understanding of FIG. 1, the following description is presented. Recombinant E. coli Arctic Express (DE3) strains grown in 50 mL of A) Luria Broth (LB) and B) Terrific Broth (TB), the protein extract fractions were separated into soluble (sol) and insoluble (ins) and analyzed on SDS-PAGE and Western blot using anti-His antibody for the detection of Zv-ΔNS1. The cultures were induced to an OD˜2.0 with IPTG and kept under agitation in flasks stirred at a temperature of 16° C. for 18 hours. C) E. coli BL21 (DE3) recombinant strains grown in LB, TB, and 2×HKSII, the protein extract fractions were separated into soluble (sol) and insoluble (ins) and analyzed in SDS-PAGE. Cultures were induced under the same conditions previously reported but maintained at 16° C. for 18 hours. D). For comparison between the clones, 5 μg of the soluble protein extract of Arctic express and E. coli BL21 (DE3) grown in TB for 18 hours were separated by SDS-PAGE and stained with Coomassie blue.

In order to increase the production of soluble Zv-ΔNS1 protein, 14 environments were tested where A (Temperature ° C.), B (Time h), and C (MM inductor concentration) varied, verifying Growth (DCW g/L) and Product (mg/L) for each set of variables. Table 1 presents the results for each tested environment.

TABLE 1

| | Variables | | | | |
|---|---|---|---|---|---|
| | A: | B: | C: IPTG | Responses | |
| | Temperature | Time | concentration | Culture | Product |
| Run | (° C.) | (h) | (mM) | (DCW g/L) | (mg/L) |
| 1 | 12.2 | 18.0 | 0.7 | 2.0 | 229 |
| 2 | 30.0 | 24.0 | 0.4 | 2.8 | 51 |
| 3 | 20.0 | 7.9 | 0.7 | 3.3 | 343 |
| 4 | 16.0 | 12.0 | 0.4 | 2.2 | 168 |
| 5 | 34.8 | 18.0 | 0.7 | 2.6 | 5 |
| 6 | 30.0 | 24.0 | 1.0 | 3.0 | 91 |
| 7 | 30.0 | 12.0 | 1.0 | 3.6 | 270 |
| 8 | 16.0 | 12.0 | 1.0 | 2.2 | 131 |
| 9 | 23.0 | 18.0 | 1.2 | 4.9 | 360 |
| *10 | 23.0 | 18.0 | 0.7 | 6.4 | 802 |
| 11 | 16.0 | 24.0 | 1.0 | 6.7 | 524 |
| 12 | 23.0 | 18.0 | 0.2 | 7.2 | 305 |
| *13 | 23.0 | 18.0 | 0.7 | 7.7 | 1073 |
| *14 | 23.0 | 18.0 | 0.7 | 7.1 | 836 |
| 15 | 23.0 | 28.1 | 0.7 | 7.0 | 187 |
| 16 | 16.0 | 24.0 | 0.4 | 5.8 | 507 |
| 17 | 30.0 | 12.0 | 0.4 | 3.6 | 179 |
| *18 | 23.0 | 18.0 | 0.7 | 7.3 | 1057 |
| *19 | 23.0 | 18.0 | 0.7 | 7.9 | 994 |

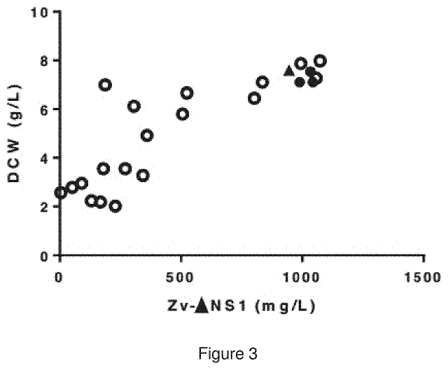

Three tests were carried out under the best conditions with a focus on maximizing growth and producing soluble Zv-ΔNS1. In the tests performed, an average of 7.25±0.25 DCW g/L and 1,022±29 mg/L of Zv-ΔNS1 was obtained. (FIG. 3). Zv-ΔNS1 growth and production were analyzed in triplicates using the best conditions obtained.

### Example II—Production of Soluble Zv-ΔNS1 Protein in a 6-L Batch Bioreactor

### Bioreactor Configuration

The best conditions identified in shaken flask experiments were applied to the Bioreactor (capacity of up to 10 L) (BioStat C-Plus, Sartorius) in single batch cultivation using 6-L of TB-kan as a culture medium and added the defoaming agent PPG 0.03%. The inoculum for the bioreactor was prepared as described below: An Erlenmeyer containing 100 mL of the same medium was grown throughout the previous night and used to inoculate the bioreactor to an initial OD of 0.1. Dissolved oxygen (pO₂) was maintained at a saturation of 30% and pH of 7.2, controlled by the automatic addition of phosphoric acid or ammonium hydroxide. The culture was maintained at a temperature of 37° C. until OD reached 2.0 when the temperature was lowered to reach 21° C. and the IPTG inducer was added at a final concentration of 0.7 mM. Cell growth was measured using regular measurements after inoculation.

The samples were also used to determine protein production and solubility of the latter.

The aliquots used to determine the optimal time for the production of soluble Zv-ΔNS1. Recombinant protein production can be observed by SDS-PAGE as early as 2 hours after induction in both soluble and insoluble fractions. Soluble Zv-ΔNS1 production reaches its maximum between 10-12 hours after induction when the optical density (OD) reaches virtually the maximum value (OD=16.3), but the Zv-ΔNS1 protein begins to concentrate in the insoluble fraction after 13 hours of induction.

In FIG. 2 it is possible to identify that the soluble and insoluble fractions were separated by SDS-PAGE and stained by Coomassie blue R-250. Aliquots are extracted after induction to determine the optical density (OD).

In bioreactor large-scale production the time condition of induction seems to be related to the dissolved oxygen and pH parameters, which are kept constant in the process of the invention (30% pO₂ and pH=7.2). In one embodiment, it turns out that even better results can be obtained with the use of phosphoric acid instead of ammonium hydroxide to avoid the tendency to alkalinize the medium.

### Example III—ΔNS1 Protein Purification Process

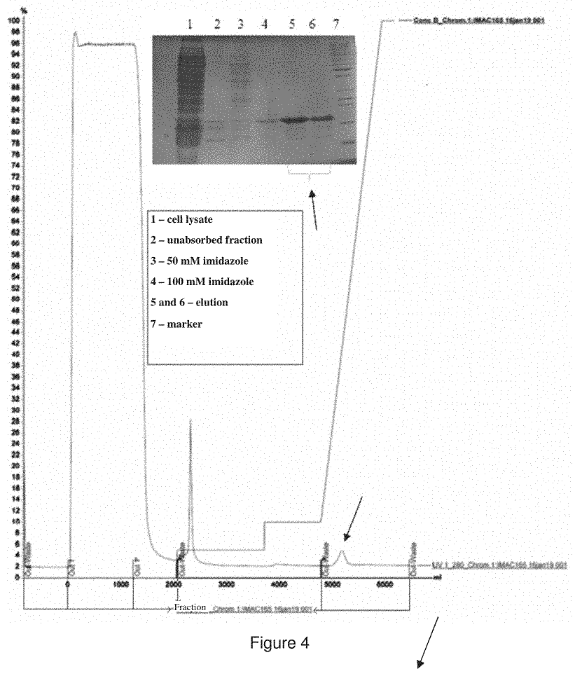

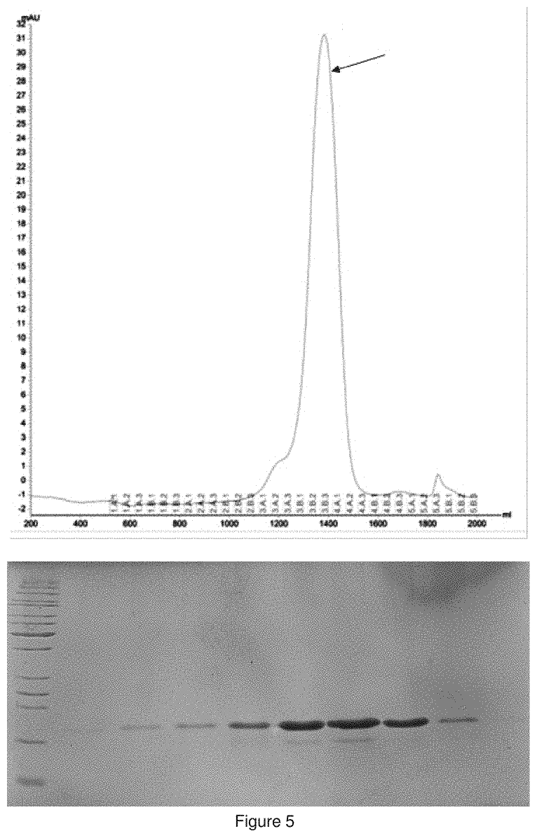

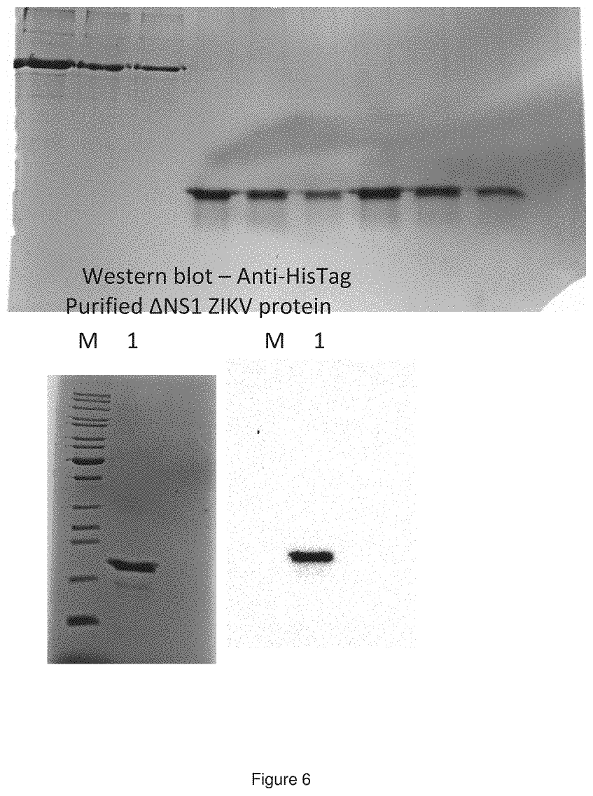

100 g of cells obtained in the bioreactor were resuspended in lysis buffer: 20 mM bis-tris propane pH 7.5, 0.5 M NaCl, 0.1% X-100 triton, 1 mM phenylmethylsulphonyl fluoride (inhibitor of proteases). Cell rupture was performed in a continuous high-pressure homogenizer, at 600 bar and 1 L/min for 8 min. Cell debris were removed by centrifugation at 17,000 g for 2 h and the supernatant applied to metal affinity chromatography loaded with Ni⁺² previously equilibrated with 10 mM bis-tris propane buffer pH 8.5, 0.5 M NaCl. Elution was carried out with an increasing gradient of imidazole (FIG. 4). The fraction containing deltaNS1 was then subjected to molecular exclusion chromatography (FIG. 5). This process made it possible to obtain 70 mg of purified protein with >95% purity (FIG. 6).

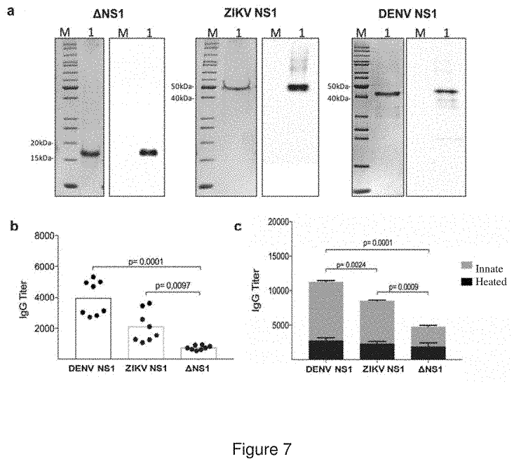

The purified protein reacted with Zika positive human serum with little cross-reactivity with Dengue positive serum, while the heat-denatured protein was not able to distinguish Zika and Dengue sera, thereby showing the importance of obtaining deltaNS1 in the soluble form (FIG. 7).

### Example IV—Use in Kit Preparation

The present invention also discloses the product obtained by the process, that is, the recombinant protein produced by the process of the invention has advantageous characteristics for use as a result of the process for obtaining it, notably with regard to folding and the immunological characteristics suitable for the development of serological tests to detect the Zika virus.

In one embodiment the product obtained by the process of the invention is used in the preparation of a kit for the in vitro diagnosis of the Zika virus. Said kit comprises: a recombinant protein obtained by the process of the invention; and a means for visualizing the interaction of said recombinant protein with an antigen or antibody that binds to said recombinant protein. In one embodiment, the kit is used in the following manner/steps:

(i) contacting a recombinant protein consisting of SEQ ID No 1, obtained by the process of the invention, with a sample of biological fluid;

(ii) providing conditions for said recombinant protein to bind to a specific antibody against it, present in said sample; and

(iii) detecting the interaction of said recombinant protein with said antibody.

Those skilled in the art know that several approaches to the detection of antibody protein binding can be used, including color labeling or other methods known in the art.

Those skilled in the art will immediately value the knowledge disclosed herein and will know that, based on the inventive concept and the examples now detailed, they will be able to materialize the inventive concept in equivalent ways, and should be understood as within the scope of the attached claims.

1. Process for the production of a recombinant fragment of the c-terminal region of the Flavivirus NS1 non-structural protein, said process being characterized in that it comprises the following steps:
(i) obtaining a host cell that comprises a flavivirus NS1 non-structural protein expression system;
(ii) cultivating the host cells at a temperature of 16 to 30° C. from 8 to 24 hours.

2. Process, according to claim 1 characterized in that the expression of the NS1 non-structural protein is induced by an inducer.

3. Process, according to claim 2 characterized in that the inductor is selected from IPTG and/or lactose, in which the concentration of the inductor is between 0.4 to 1 mM.

4. Process, according to claim 1 characterized in that the cultivation time is 10 to 12 hours for cultivation in a bioreactor or 18 to 24 hours for cultivation in flask.

5. Process, according to claim 1 characterized in that the culture medium comprises LB, TB and/or 2×HKSII in which the host cell would be Escherichia coli.

6. Process, according to any of the preceding claims characterized in that is carried out in one or more of the following conditions:
temperature of 21° C.;
induction of the expression of the gene of interest made with 0.7 mM of IPTG;
host microorganism selected from Escherichia coli Arctic Express (DE3) and BL21 (DE3).

7. Process, according to claim 6, characterized in that the host cell is BL21 (DE3).

8. Process, according to any one of claims 1 to 7 characterized in that the product obtained presents solubility in water above 60% in relation to the total amount of soluble and insoluble protein.

9. Process, according to any of the preceding claims characterized in that the concentration of the final product obtained is greater than 0.5 g/L.

10. Process, according to any one of the preceding claims, characterized in that the recombinant fragment of the c-terminal region of the flavivirus NS1 non-structural soluble protein has 70% or more identity to SEQ ID NO 1.

11. Process according to any one of claims 1 to 9 characterized in that the recombinant fragment of the c-terminal region of the flavivirus NS1 non-structural soluble protein is SEQ ID No 1.

12. Process according to claim 11 characterized in that the nucleic acid comprised in the host cell is SEQ ID No 2.

13. Process for the purification of the recombinant fragment of the c-terminal region of the Flavivirus NS1 non-structural protein, produced according to the process as defined in any one of claims 1 to 12, characterized in that it comprises the steps of:
(a) cell lysis;
(b) centrifugation;
(c) metal affinity chromatography; and
(d) molecular exclusion chromatography for the separation of the recombinant protein from the c-terminal region of the Flavivirus NS1 non-structural protein.

14. Process according to claim 13 characterized in that the purification process is carried out under one or more of the following conditions:
(a) cell disruption was performed in a continuous high-pressure homogenizer, at 600 bar and 1 L/min for 8 min;
(b) centrifugation at 17,000 g for 2 h;
(c) Ni⁺² charged metal affinity chromatography previously equilibrated with 10 mM bis-tris propane buffer pH 8.5, 0.5 M NaCl; and/or
(d) elution performed with an increasing gradient of imidazole, followed by molecular exclusion chromatography.

15. Product characterized in that it is obtained by the process defined in any one of claims 1 to 14.

16. Use of the product as defined in claim 15, characterized in that it is for the preparation of a kit for identifying the interaction of the recombinant fragment of the c-terminal region of the NS1 non-structural protein with antibodies against flavivirus.

17. Method of detecting interaction between antibodies generated after infection by ZIKV, DENV, or other flaviviruses characterized in that it comprises the steps of:
(i) contacting a recombinant fragment of the c-terminal region of the flavivirus NS1 non-structural soluble protein, obtained by the process as defined in any one of claims 1 to 16, with a sample of biological fluid ex vivo;
(ii) providing conditions for said recombinant protein to bind to a specific antibody against the same, present in said sample;
(iii) visualizing or detecting the interaction of said recombinant protein with said antibody;
(iv) comparing the results of said visualization and/or detection with a standard.

18. Method of diagnosis of diseases caused by flavivirus characterized in that it comprises the steps of:
(i) contacting a recombinant fragment of the c-terminal region of the flavivirus NS1 non-structural soluble protein, obtained by the process as defined in any one of claims 1 to 16, with a sample of biological fluid;
(ii) providing conditions for said recombinant protein to bind to a specific antibody against it, present in said sample;
(iii) visualizing the interaction of said recombinant protein with said antibody;
(iv) diagnosing whether the biological fluid sample is, or has been, infected with a flavivirus or not.

19. Method of diagnosis as defined in claim 17 or 18, characterized in that the flavivirus is selected from the group comprising yellow fever; dengue 1, dengue 2, dengue 3 and dengue 4, Zika virus, West Niles virus, Saint Louis encephalitis virus, Murray Valley encephalitis virus, tick encephalitis.
